(19) United States
(12) Patent Application Publication
(10) Pub. No.: US 2017/0072628 A1
(43) Pub. Date: Mar. 16, 2017

(54) NOZZLE FOR SELECTIVELY GENERATING EITHER PLASMA OR ULTRAVIOLET RADIATION

(71) Applicant: Drexel University, Philadelphia, PA (US)

(72) Inventors: Wei Sun, Cherry Hill, NJ (US); Qudus Hamid, Philadelphia, PA (US); Chengyang Wang, Bengbu (CN)

(73) Assignee: Drexel University, Philadelphia, PA (US)

(21) Appl. No.: 15/310,994

(22) PCT Filed: May 27, 2015
(86) PCT No.: PCT/US15/32747

§ 371 Date: Nov. 14, 2016

Related U.S. Application Data
(60) Provisional application No. 62/003,768, filed on May 28, 2014.

(51) Int. Cl. B29C 67/00; H05G 2/00; B29C 35/08; C12N 5/00; B33Y 30/00; B33Y 50/02; B33Y 70/00; H05H 1/24; B33Y 10/00

(52) U.S. Cl. CPC B29C 67/007; H05H 1/2406; H05G 2/006; B29C 67/0088; B29C 35/0866; B29C 35/0805; B33Y 10/00; B33Y 30/00; B33Y 50/02; B33Y 70/00; C12N 5/0068; H05H 2001/2456; B29C 2035/0827; B29K 2995/0056; B29L 2031/752

(57) ABSTRACT

One aspect of the invention provides a method of generating ultraviolet radiation. The method includes: introducing a mixture of nitrogen gas and helium gas into a dielectric barrier discharge device including a high voltage electrode and a ground electrode separated by a gap and a dielectric; and applying suitable electricity to the high voltage electrode, thereby generating ultraviolet radiation. Another aspect of the invention provides a plasma- and ultraviolet-generating nozzle including: a dielectric barrier discharge device; and a controller programmed to control flows of gas into the dielectric barrier discharge device and application of suitable electricity to the dielectric barrier discharge device to selectively generate either plasma or ultraviolet radiation. Another aspect of the invention provides a printing system including: a motion control system; and the plasma- and ultraviolet-generating nozzle as described herein coupled to the motion control system.

### CROSS-REFERENCE TO RELATED APPLICATION

This application claims priority to U.S. Provisional Patent Application Ser. No. 62/003,768, filed May 28, 2014. The entire content of this application is hereby incorporated by reference herein.

### STATEMENT AS TO RIGHTS TO INVENTIONS MADE UNDER FEDERAL SPONSORED RESEARCH AND DEVELOPMENT

This invention was made with government support under Grant No. CMMI-1030520 awarded by the National Science Foundation. The U.S. Government has certain rights in this invention.

### BACKGROUND OF THE INVENTION

The use of animal and human models is limited by the feasibility of testing protocols, availability, and ethical concerns. As a result, monolayer cell cultures are used to investigate potential anti-cancer agents. Monolayer investigations are limited because these two-dimensional (2D) models give very little feedback on the effects of the micro-environment on chemotherapeutic and the heterogeneity of the tumor.

### SUMMARY OF THE INVENTION

One aspect of the invention provides a method of generating ultraviolet radiation. The method includes: introducing a mixture of nitrogen gas and helium gas into a dielectric barrier discharge device including a high voltage electrode and a ground electrode separated by a gap and a dielectric; and applying suitable electricity to the high voltage electrode, thereby generating ultraviolet radiation.

This aspect of the invention can have a variety of embodiments. The ultraviolet radiation can have a wavelength between about 300 nm and about 400 nm. The dielectric barrier discharge device can be a cylindrical dielectric barrier discharge device. The suitable electricity can have a voltage between about 4 kV and about 16 kV. The suitable electricity can have a frequency between about 20 kHz and about 30 kHz. The mixture can includes about 90% helium gas and about 10% nitrogen gas by volume.

Another aspect of the invention provides a plasma- and ultraviolet-generating nozzle including: a dielectric barrier discharge device; and a controller programmed to control flows of gas into the dielectric barrier discharge device and application of suitable electricity to the dielectric barrier discharge device to selectively generate either plasma or ultraviolet radiation.

This aspect of the invention can have a variety of embodiments. The controller can be programmed to introduce a mixture of nitrogen gas and helium gas into the dielectric barrier discharge device prior to application of the suitable electricity in order to generate ultraviolet radiation. The mixture can include about 90% helium gas and about 10% nitrogen gas by volume.

The controller can be programmed to introduce a mixture of at least one noble gas and oxygen gas into the dielectric barrier discharge device prior to application of the suitable electricity in order to generate plasma. The at least one noble gas can include helium. The mixture can include about 95% of the at least one noble gas and about 5% oxygen gas by volume.

Another aspect of the invention provides a printing system including: a motion control system; and the plasma- and ultraviolet-generating nozzle as described herein coupled to the motion control system.

This aspect of the invention have a variety of embodiments. The printing system can further include: a polymer nozzle adapted and configured to print a photocurable polymer and a biologics nozzle adapted and configured to deposit one or more cells. The printing system can further include a system controller programmed to control operation of the motion control system, the plasma- and ultraviolet-generating nozzle, the polymer nozzle, and the biologic nozzle. The system controller can be programmed to: actuate the motion control system to move the nozzles to a first specified location; actuate the polymer nozzle to apply a photocurable polymer at the first specified location; actuate the plasma- and ultraviolet-generating nozzle to generate ultraviolet radiation to cure the photocurable polymer at the first specified location; actuate the plasma- and ultraviolet-generating nozzle to generate plasma at a second specified location adjacent to the first specified location; and actuate the biologics nozzle to deposit one or more cells to the second specified location.

Another aspect of the invention provides a method of generating a substrate. The method includes: applying a photocurable polymer at the first specified location; actuating the plasma- and ultraviolet-generating nozzle as described herein to generate ultraviolet radiation to cure the photocurable polymer at the first specified location; actuating the plasma- and ultraviolet-generating nozzle to generate plasma at a second specified location adjacent to the first specified location; and depositing one or more cells at the second specified location.

Another aspect of the invention provides a substrate fabricated according to the methods described herein.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views and wherein:

FIG. 1 depicts a model of a system according an embodiment of the invention along with detail views of the cell and a UV/plasma nozzle according to an embodiment of the invention;

FIG. 2 depicts a flow chart of the integrated system with each of its major components outlined with color-coded dashed lines according to an embodiment of the invention;

FIG. 3 is a schematic of the printer's head and the process information pipeline for biologics printing according to an embodiment of the invention;

FIG. 4 is a schematic view of the dual micro-plasma and UV nozzle and the flow chart of the patterning tool-path creation from the designed pattern according to an embodiment of the invention;

FIG. 5 depicts a method 500 of generating ultraviolet radiation according to an embodiment of the invention;

FIG. 6 depicts a printing system according to an embodiment of the invention;

FIG. 7 shows the orientation of the three-dimensional spatial control motion arms according to according to an embodiment of the invention;

FIG. 8 depicts a method of generating a substrate according to an embodiment of the invention; and

FIG. 9 depicts a cross-section of a 3D substrate according to an embodiment of the invention.

### DEFINITIONS

The instant invention is most clearly understood with reference to the following definitions:

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION OF THE INVENTION

Cancer progression and invasion into surrounding normal tissue are influenced through the reciprocal interactions with host stromal cells including fibroblasts, endothelial cells, and macrophages. Cancer expansion and invasion cannot be studied in conventional 2D co-culture model. Additionally, a tumor normally expands within a confining environment, which leads to high stresses in both the tumor tissue and the surrounding tissue. For example, breast adenocarcinoma cells under compressive strains mimicking the growing cells within a confining environment showed up-regulation of genes related to invasion and metastasis.

Cancer has long been recognized as many diseases due to its difference among each patient. In addition to the patient heterogeneity, phenotypic and functional heterogeneity and plasticity within tumors and between primary tumors and metastases has been brought into tumor understanding over the past few decades. One possible cause of the heterogeneity within tumors is the intercellular genomic instability that leads to a branched evolution in tumors. The branched evolution has now been observed among multiple tumor types, including adenoma-to-carcinoma transition of the colon, childhood acute lymphoblastic leukemia (ALL), chronic lymphoblastic leukemia (CLL), pancreatic cancer, and breast cancer. Moreover, the heterogeneity in the micro-environment that include cancer-associated fibroblast, immune cells, vascular network and extracellular matrix, is another cause of the heterogeneous hierarchy. Those heterogeneities in tumorigenesis result in a tumor that is a complex of distinct subpopulations of tumorigenic cancer cells, their non-tumorigenic progeny, and supporting cells. This heterogeneous hierarchy has been denominated as cancer stem-cell model and has been demonstrated in various tumor types including acute myeloid leukemia (AML), chronic myeloid leukemia (CIVIL), breast cancer, glioblastoma, colorectal cancer, pancreatic cancer, and ovarian cancer. Due to the complexity of such heterogeneity, clinical assessment of anticancer drugs poses to several practical challenges because of the limitations in current transplantation cancer model. The need for development in preclinical model system essentially increases along with the notion of personalized drugs that seek to choose the efficient drug for each patient individually.

Aspects of the invention presented herein (1) eliminate the need for masks by incorporating a dynamic maskless fabrication technique, (2) allow for direct surface enhancements as the model is being fabricated, (3) eliminate the need for long fabrication processes, (4) eliminate the use of toxic chemicals, and (5) allow for spatially controlled heterogeneous deposition of cells/biologics as the tissue array is being fabricated. The integrated system can develop models on a micro-scale level, thereby making investigations more economic, requiring less reagents and cells, and, above all, enabling consistency in experimental analysis to due limited interactions with the end user. Aspects of the invention are particularly useful for the development of biologically inspired devices including, but not limited to, biosensors, Lindenmayer systems, and micro-organs.

Aspects of the invention integrate several critical fabrication components utilized in the fabrication of many biological arrays/platforms. These system can include 3D spatial control, material deposition, photolithography, and plasma treatment components. The 3D spatial control component can house all components on the z-motion arm with interconnectivity to an x- and y-arm for a complete 3D motion. The material deposition component can house the biological nozzle and the photo-polymer nozzle. The biologics printer can include a cell-friendly deposition head that is placed on the motion arm and used for the spatial deposition and orientation of the cells and or biologics in the micro-channels. This component can include two or more nozzles for controlled deposition of multiple cell types. The final nozzle of the material delivery component can be a piston style nozzle that is used to drive material of higher viscosity, such as photo-curable polymers. The photolithographic and plasma treatment systems can be coupled together into one nozzle as discussed in greater detail below. The nozzle integration can utilizes the composition of various gases and high electric fields to generate both (1) ultraviolet light for the crosslinking of the photoresist immediately after deposition and, (2) prior to cell deposition, oxygen-based plasma for surface functionalization.

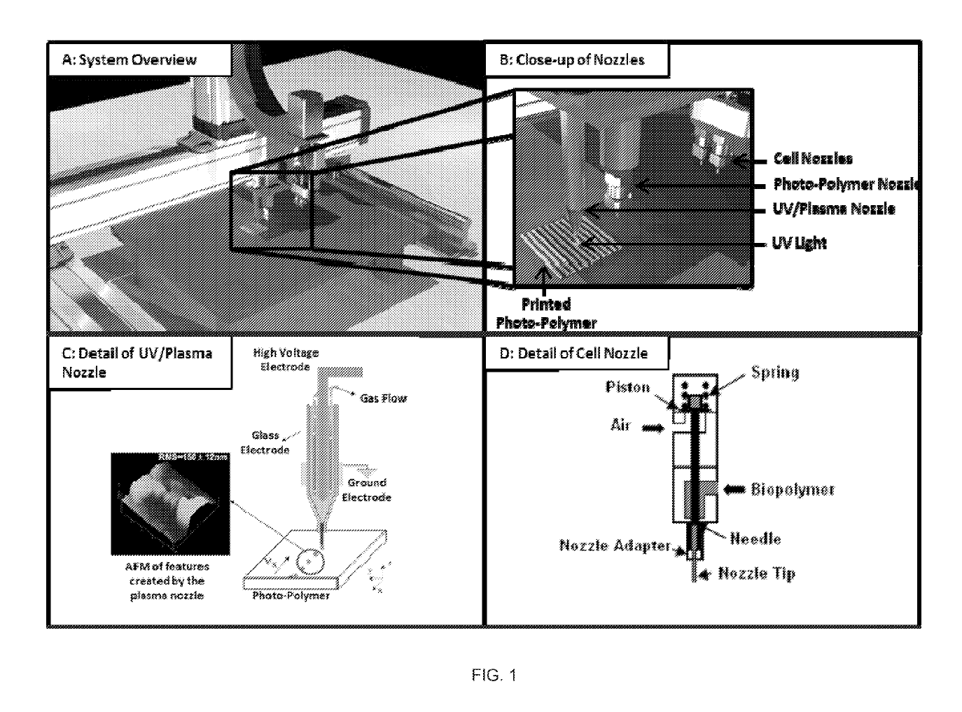

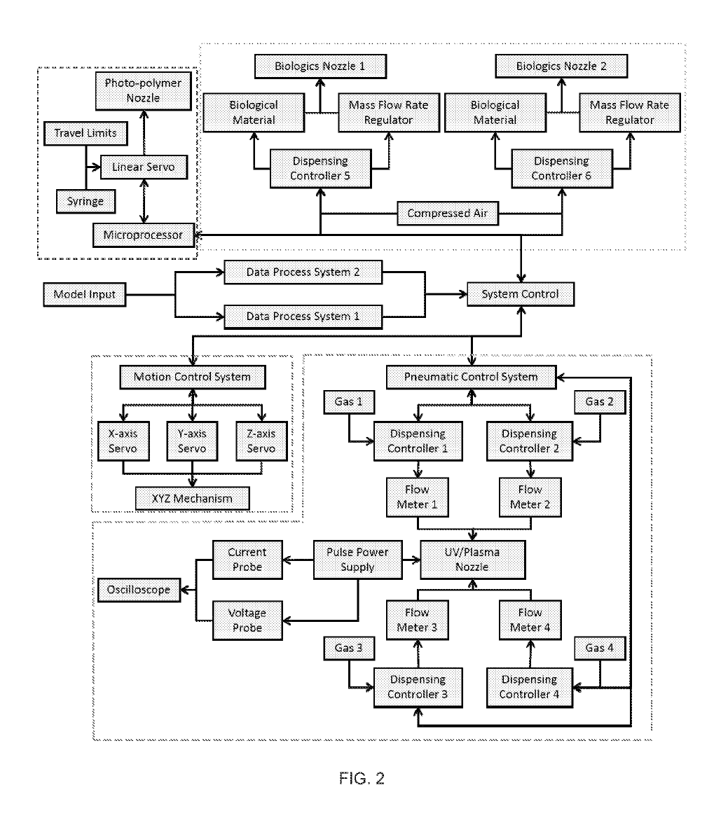

FIG. 1 shows a model of a system according an embodiment of the invention along with detail views of the cell and a UV/plasma nozzle. FIG. 2 presents a flow chart of the integrated system with each of its major components outlined with color-coded dashed lines.

### Biologics Printer

The biologics component can be built on a CAD/CAM platform that can integrate with the three-dimensional spatial control component. The biologics printer can operate at cell-friendly conditions of room temperature and low pressure conditions. Coupled with the spatial control component, the biologics printer can deposit multiple cell types and bioactive factors in controlled amounts with precise spatial positioning. The printer can utilize a micro-valve nozzle. This nozzle enables the printer to deposit a wide range of solutions with a wide range of material and biological properties. This system eliminates human errors and provides its end users with precision control during fabrication procedures. The biologics deposition component is capable of depositing heterogeneous materials, cell types, and biological factors in a controlled and reproducible manner. Cell printing is considered to be an effective biofabrication tool to assemble biologics.

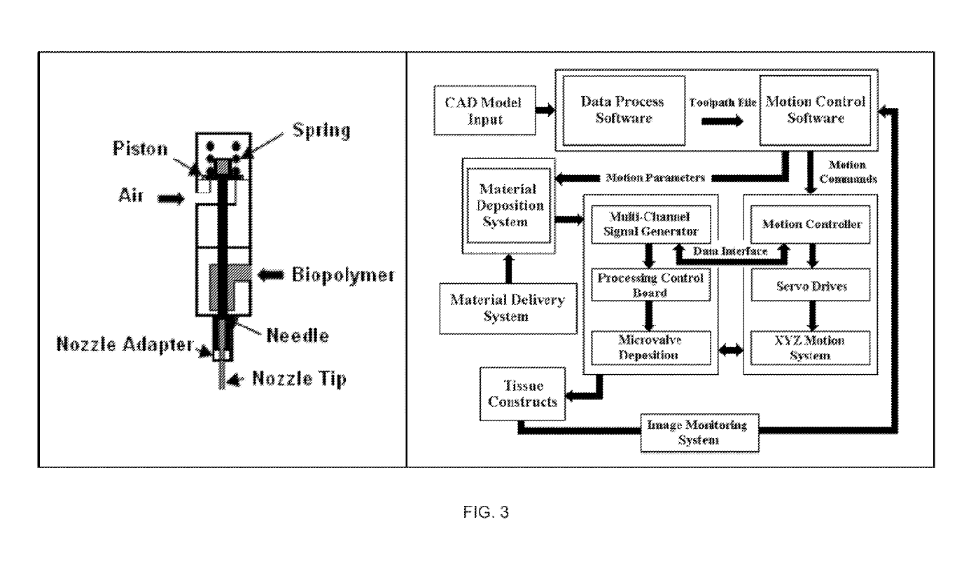

FIG. 3 provides a schematic of the printer's head and the process information pipeline for biologics printing. Some embodiments include two pneumatic micro-valve nozzles integrated for the deposition of biologics and biomaterials, respectively.

### Integration of a Plasma and Ultraviolet Generation in a Single Nozzle

Embodiments of the invention integrate plasma and ultraviolet (UV) generation into a single nozzle, thereby enabling the generation of micron scale patterns on a substrate without using chemical solvents and masks or master stamps.

The same nozzle generates both plasma and UV by changing the gas types and flow composition within the nozzle along with the corresponding process parameter for generation of the desired ignition. UV generation involves the ignition of gases that produces UV light (e.g., having a wavelength between about 300 nm and about 400 nm). Plasma generation is the excitation of ions that bombards the substrate's surface to manipulate its topology, surface chemistry, and/or functional groups. In embodiments of the invention, micro-plasma and UV are delivered through the dielectric barrier discharge (DBD) technique. DBDs are non-equilibrium plasmas operated under atmospheric pressure. Due to a non-equilibrium nature, DBD plasmas can generate high energy electrons at cool background gas temperatures (heavy particles). This unique character (selective high electron temperature, and low background temperature) enables rich plasma chemistry in many plasma chemical processes.

The micro-plasma and UV component can include a power supply and plasma electrode components. Micro-plasma can be generated by a pulsed power supply with variable frequency. The plasma electrode system can include a high voltage electrode coaxially inserted in a dielectric (e.g., borosilicate glass or quartz) tube and a ground electrode wrapped around the tube from the outside. The process gas (or gas mixture) can be purged through the annular gap between the coaxial electrode and the dielectric tube. When the high voltage electrode is powered to generate plasma, the gas will ignite between the electrodes and a micron-scale glow-like plasma will appear at the tip of the nozzle. Once the micro-plasma contacts the surface of biopolymer, it will change the topography and chemistry of the plasma-exposed area. When UV is generated and contacts the substrate, it will cross-link the photo-polymer. By varying operation parameters such as power, gas flow rate, gas composition, and nozzle tip diameter, it will be possible to control UV range, chemical composition, and topological features of the exposed photo-polymer.

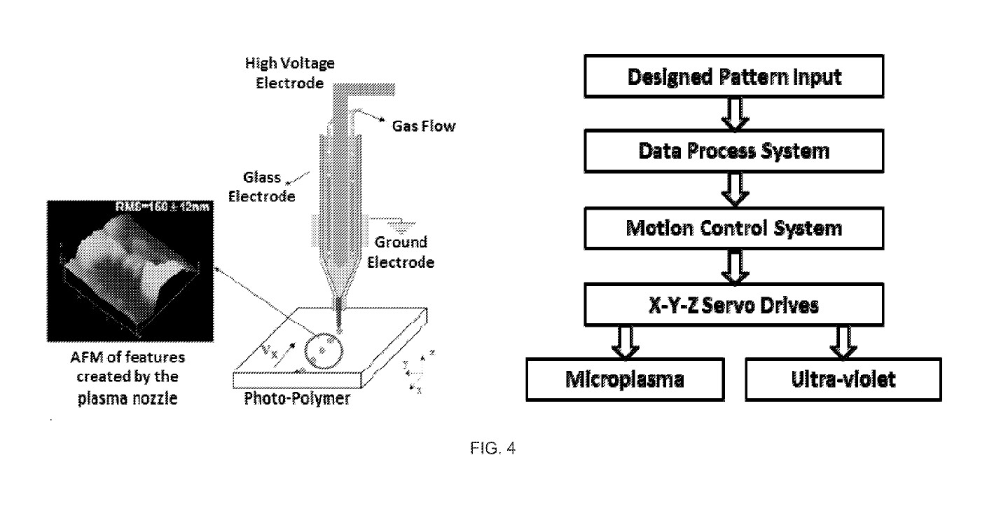

FIG. 4 shows a schematic view of a dual micro-plasma and UV nozzle and a flow chart of a patterning tool-path creation from a designed pattern.

The micro-plasma and UV component can be integrated with the three-dimensional spatial control component. This integration will give the plasma component full spatial control to create a pattern and control the shape of the pattern over the substrate. The CAD-based system converts the CAD model or designed pattern into a layered process tool path. The UV/plasma components can communicate with the computer interface (via controllers) to treat the substrate with the desired plasma or ultraviolet radiation to produce the modeled array.

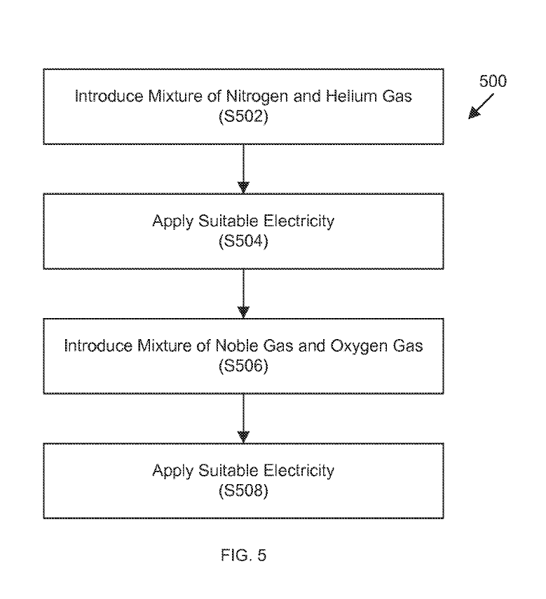

Referring now to FIG. 5, a method 500 of generating ultraviolet radiation is provided.

In step S502, a mixture of nitrogen gas and helium gas is introduced into a dielectric barrier discharge (DBD) device (e.g., a cylindrical DBD device as FIG. 1, Panel C and FIG. 4). The mixture can, but need not consist exclusively or essentially of nitrogen and helium gas. Other gases (e.g., noble gases) can be present. For example, the mixture can include about 90% helium and about 10% nitrogen by volume.

In step S504, suitable electricity is applied to the high voltage electrode of the DBD device. For example, the electricity can have a voltage between about 4 kV and about 16 kV and/or a frequency between about 20 kHz and about 30 kHz.

The electricity initiates the following chain of reactions, which emit UV radiation:

| | |
|---|---|
| He | e + He → 2e + He⁺ |
| | e + He → e + He^{m} |
| | e + He^{m} → 2e + He⁺ |
| | 2He^{m} → e + He + He⁺ |
| | e + He^{m} → e + He |
| | e + He₂^{m} → He^{m} + He |
| | He^{m} + 2He → He₂^{m} + He |
| | He⁺ + 2He → He₂⁺ He |
| N₂ | e + N₂ → 2e + N₂⁺ |
| He + N₂ | He^{m} + N₂ → e + N₂⁺ + He |
| | He₂^{m} + N₂ → e + N₂⁺ + 2He |
| | He⁺ + N₂ → N₂⁺ + He |
| | He₂⁺ + N₂ → N₂⁺ + 2He |

In step S506, a mixture of a noble gas and oxygen gas can be introduced into the DBD device in order to generate a plasma. In one embodiment, the mixture includes about 95% helium and about 5% oxygen by volume.

In step S508, suitable electricity is applied to the high voltage of the DBD device. For example, the electricity can have a voltage between about 4 kV and about 16 kV and/or a frequency between about 20 kHz and about 30 kHz.

The electricity initiates the following chain of reactions, which emit plasma radiation:

| | |
|---|---|
| He | e + He → 2e + He⁺ |
| | e + He → e + He^{m} |
| | e + He^{m} → 2e + He⁺ |
| | 2He^{m} → e + He + He⁺ |
| | e + He^{m} → e + He |
| | e + He₂^{m} → He^{m} + He |
| | He^{m} + 2He → He₂^{m} + He |
| | He⁺ + 2He → He₂⁺ + He |
| O₂ | e + O₂ → 2e + O₂⁺ |
| | e + O₂ → 2e + O₂⁺ |
| | e + O₂ → e + 2O |
| | e + O₂ → O + O⁻ |
| | e + O₂⁺ → 2O |
| | e + 2O₂ → O₂⁻ + O₂ |
| | O⁻ + O₂⁺ → O + O₂ |

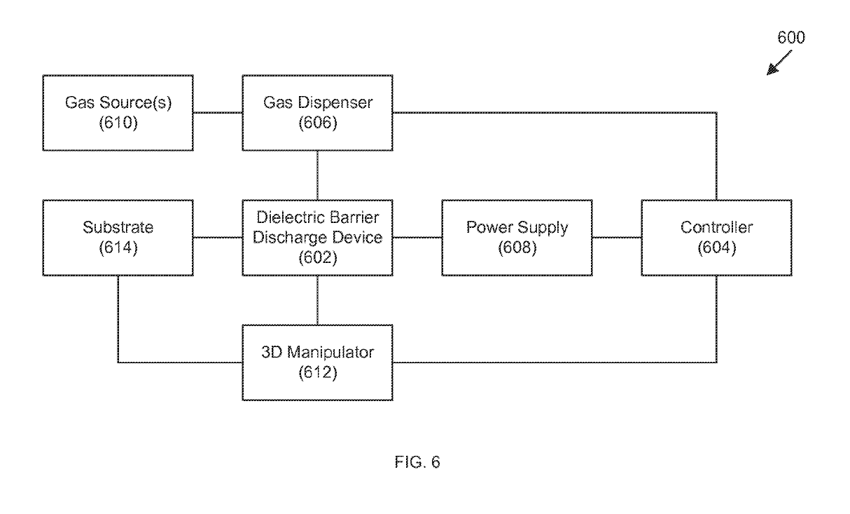

Referring now to FIG. 6, a printing system 600 is provided. System 600 includes a DBD device 602 and a controller 604. The controller 604 can be programmed to control one or more gas dispensers 606 to introduce a desired gas mixture into the DBD device 602 for a desired application (e.g., plasma or UV generation) and power supply 608 to provide suitable electricity to generate plasma or UV generation. Gas dispenser(s) 606 can include one or more valves and/or flow sensors to measure and control the flow of gases from gas sources 610 (e.g., tanks of gases such as noble gases, helium, nitrogen, oxygen, and the like). Controller 604 can also control or can be in communication or integrated with another controller for moving the DBD device 602 and/or a substrate to be printed 614.

### Integration of Three-Dimensional Spatial Control

The three-dimensional spatial control component can be integrated with other components (e.g., ultra-violet, plasma, and biologic nozzles) and can function independently of each component. The UV and plasma head can be a single, dual-function nozzle, while the biologic nozzles can be separate heads. All nozzles/print head can be independent of each other. In some embodiments, only one head is utilized at once. All print heads can be housed on the third (z-axis) motion arms. All nozzles can utilize the spatial controllers in sequential order to develop and enhance the fabricated arrays while depositing biologics into the channels. The motion system can be controlled by a proportional-integral-derivative controller (PID controller) in order to enable tuning of the entire system to function adequately given any fabrication task.

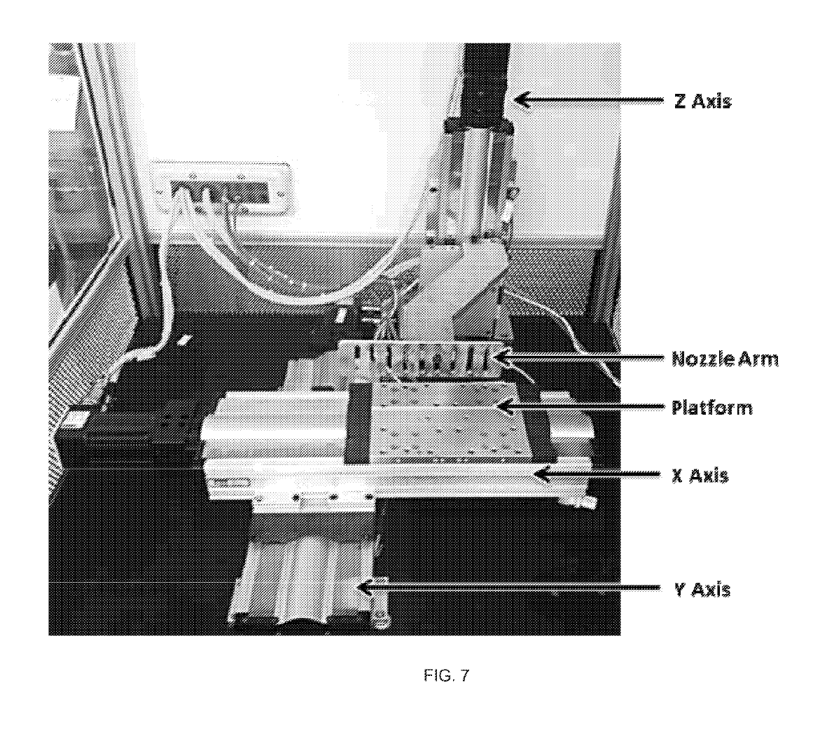

FIG. 7 shows the orientation of the three-dimensional spatial control motion arms. An interface can enable complete integration of the controllers and the fabrication of the desired model.

### Integrating Hardware Communication

The dual UV and plasma head can be controlled with pneumatic controllers along with electronic flow regulators. A controller and regulator can be used for each gas line. Each of these components can be wired to a communication hub that is connected to a computing system and function under Boolean operations. The biological heads can be wired to micro-controllers that control the drive speed with is proportional to the material mass transfer. The micro-controllers on these units can have an open-sourced firmware and can be easily integrated to many different computing languages. The three-dimensional motion arms can be independently controlled with a proportional-integral-derivative (PID) system and can then be wired to the central hub. These controllers can have an open communication port that allows for integration onto user friendly software. All controllers utilized in this system can be programmed. In addition to firmware integration and the development of GUI to operate all hardware independently, the GUI can be able to read simple CAD models and build the inputted model. The GUI can utilize .stl (STereoLithography) CAD files. These files represent the geometric configuration of the CAD model in thin layers. Each layer, when stacked together forms a three-dimensional model. This file will be imported to the GUI, which will then utilize the geometric information for the fabrication of the desired model.

### Method of Generating a Substrate

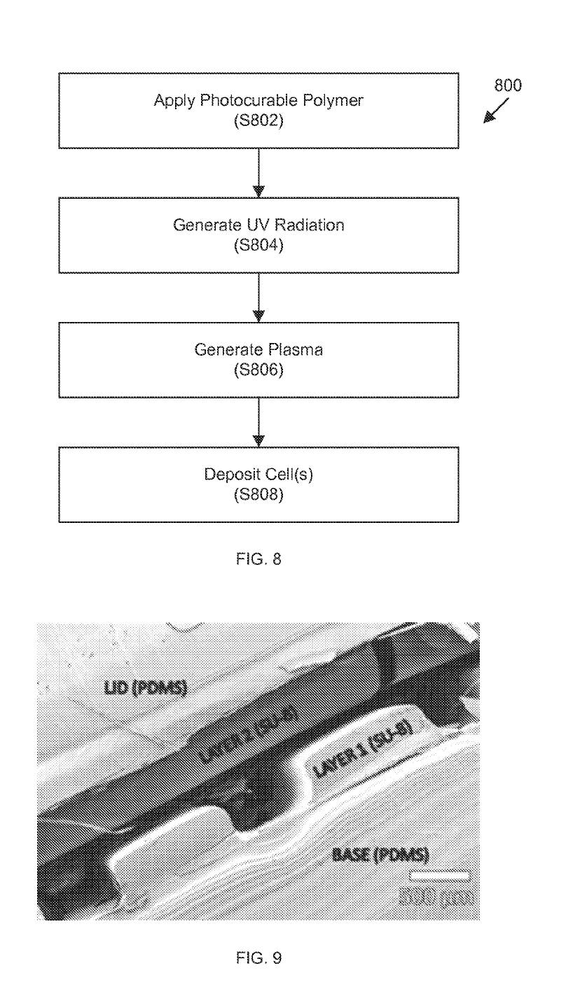

Referring now to FIG. 8, a method 800 of generating a substrate is provided.

In step S802, a photocurable polymer is applied at a first specified location. In one embodiment, the photocurable polymer is SU-8.

In step S804, the plasma- and ultraviolet-generating nozzle as described herein is actuated to generate ultraviolet radiation to cure the photocurable polymer at the first specified location.

In step S806, the plasma- and ultraviolet-generating nozzle as described herein is actuated to generate plasma at a second specified location adjacent to the first specified location. For example, the photocurable polymer can define a plurality of walls or ridges and plasma can be used to treat valleys or channels between the walls or ridges.

In step S808, one or more cells are deposited in the second specified location, which was treated with plasma in the previous step.

### Substrates

Referring now to FIG. 9, a scanning electron micrograph of a cross-section of a 3D substrate is provided. Although the substrate shown was generated using a 3D printer having a separate UV head, the combined plasma- and ultraviolet-generating nozzle described herein would be capable of fabricating the same or similar structure.

Implementation in Computer-Readable Media and/or Hardware

The methods described herein can be readily implemented in software that can be stored in computer-readable media for execution by a computer processor. For example, the computer-readable media can be volatile memory (e.g., random access memory and the like) non-volatile memory (e.g., read-only memory, hard disks, floppy disks, magnetic tape, optical discs, paper tape, punch cards, and the like).

Additionally or alternatively, the methods described herein can be implemented in computer hardware such as an application-specific integrated circuit (ASIC).

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

Additionally, Applicant(s) note and hereby incorporate by reference herein the following publications by Applicant(s) describing 3D printing devices: U.S. Pat. No. 8,639,484 and U.S. Patent Application Publication Nos. 2006/195179, 2008/020049, 2008/145639, 2009/263849, 2011/136162, 2011/165646, 2011/177590, and 2012/080814.

1. A method of generating ultraviolet radiation, the method comprising:
introducing a mixture of nitrogen gas and helium gas into a dielectric barrier discharge device including a high voltage electrode and a ground electrode separated by a gap and a dielectric; and
applying suitable electricity to the high voltage electrode, thereby generating ultraviolet radiation.

2. The method of claim 1, wherein the ultraviolet radiation has a wavelength between about 300 nm and about 400 nm.

3. The method of claim 1, wherein the dielectric barrier discharge device is a cylindrical dielectric barrier discharge device.

4. The method of claim 1, wherein the suitable electricity has a voltage between about 4 kV and about 16 kV.

5. The method of claim 1, wherein the suitable electricity has a frequency between about 20 kHz and about 30 kHz.

6. The method of claim 1, wherein the mixture includes about 90% helium gas and about 10% nitrogen gas by volume.

7. A plasma- and ultraviolet-generating nozzle comprising:
a dielectric barrier discharge device; and
a controller programmed to control flows of gas into the dielectric barrier discharge device and application of suitable electricity to the dielectric barrier discharge device to selectively generate either plasma or ultraviolet radiation.

8. The plasma- and ultraviolet-generating nozzle of claim 7, wherein the controller is programmed to introduce a mixture of nitrogen gas and helium gas into the dielectric barrier discharge device prior to application of the suitable electricity in order to generate ultraviolet radiation.

9. The plasma- and ultraviolet-generating nozzle of claim 8, wherein the mixture includes about 90% helium gas and about 10% nitrogen gas by volume.

10. The plasma- and ultraviolet-generating nozzle of claim 7, wherein the controller is programmed to introduce a mixture of at least one noble gas and oxygen gas into the dielectric barrier discharge device prior to application of the suitable electricity in order to generate plasma.

11. The plasma- and ultraviolet-generating nozzle of claim 10, wherein the at least one noble gas includes helium.

12. The plasma- and ultraviolet-generating nozzle of claim 10, wherein the mixture includes about 95% of the at least one noble gas and about 5% oxygen gas by volume.

13. A printing system comprising:
a motion control system; and
the plasma- and ultraviolet-generating nozzle of claim 7 coupled to the motion control system.

14. The printing system of claim 13, further comprising:
a polymer nozzle adapted and configured to print a photocurable polymer; and
a biologics nozzle adapted and configured to deposit one or more cells.

15. The printing system of claim 14, further comprising:
a system controller programmed to control operation of the motion control system, the plasma- and ultraviolet-generating nozzle, the polymer nozzle, and the biologic nozzle.

16. The printing system of claim 15, wherein the system controller is programmed to:
actuate the motion control system to move the nozzles to a first specified location;
actuate the polymer nozzle to apply a photocurable polymer at the first specified location;
actuate the plasma- and ultraviolet-generating nozzle to generate ultraviolet radiation to cure the photocurable polymer at the first specified location;
actuate the plasma- and ultraviolet-generating nozzle to generate plasma at a second specified location adjacent to the first specified location; and
actuate the biologics nozzle to deposit one or more cells to the second specified location.

17. A method of generating a substrate, the method comprising:
applying a photocurable polymer at the first specified location;
actuating the plasma- and ultraviolet-generating nozzle of claim 11 to generate ultraviolet radiation to cure the photocurable polymer at the first specified location;
actuating the plasma- and ultraviolet-generating nozzle to generate plasma at a second specified location adjacent to the first specified location; and
depositing one or more cells at the second specified location.

18. A substrate fabricated according to the method of claim 17.
